(19) United States
(12) Patent Application Publication
(10) Pub. No.: US 2025/0228751 A1
(43) Pub. Date: Jul. 17, 2025

(54) APPLICATION OF CALCIUM ALUMINATE IN PRE-MIXED DENTAL FILLING MATERIALS, PRE-MIXED DENTAL FILLING MATERIALS AND THEIR PREPARATION METHODS

(71) Applicant: KUNMING QINGCHENG HEALTHCARE TECH LTD., Kunming (CN)

(72) Inventors: Lei ZHANG, Kunming (CN); David LUK, Kunming (CN); Yu SUN, Kunming (CN); Tuojiang WU, Kunming (CN); Yang SONG, Kunming (CN); Kewang LU, Kunming (CN); Xiaojun SHI, Kunming (CN)

(21) Appl. No.: 18/810,793

(22) Filed: Aug. 21, 2024

(30) Foreign Application Priority Data
Jan. 12, 2024 (CN) 202410046776.0

(51) Int. Cl. A61K 6/86; A61K 6/70; A61K 6/864

(52) U.S. Cl. CPC A61K 6/86; A61K 6/70; A61K 6/864

(57) ABSTRACT

The present disclosure discloses the application of calcium aluminate in pre-mixed dental filling materials, the pre-mixed dental filling materials themselves, and their preparation methods. In the pre-mixed dental filling materials of the present disclosure, calcium aluminate is used as the primary hydraulic setting material, supplemented by calcium oxide as a curing accelerator and expansion agent, and may also include calcium pyrophosphate. The resulting material is a biologically hydraulic paste with excellent injectability. This material remains fluid under sealed conditions and hardens upon hydration when placed in a physiological environment and exposed to physiological fluids. The disclosure leverages the excellent biocompatibility and bioactivity of calcium aluminate to prepare pre-mixed aluminate hydraulic dental filling materials, which can be used for medical and dental applications, including pulp capping, root canal therapy, and hard tissue repair.

### CROSS-REFERENCE TO RELATED APPLICATIONS

This application claims priority to Chinese patent application No. 202410046776.0, filed on Jan. 12, 2024, the content of which is hereby incorporated by reference in its entirety.

### TECHNICAL FIELD

The present disclosure relates to the field of dental technology, specifically to the application of calcium aluminate in pre-mixed dental filling materials, pre-mixed dental filling materials, and their preparation methods.

### BACKGROUND

Dental filling materials have wide applications in clinical dentistry and are classified functionally into dental bonding filling materials, dental restorative filling materials, and dental filling materials that provide protective functions. Currently, the most commonly used material in dental root canal filling materials is Mineral Trioxide Aggregate (MTA), a calcium silicate-based mineral trioxide aggregate. MTA is a dual-component material consisting of tricalcium silicate and dicalcium silicate, which requires mixing immediately before use. Pre-mixed root canal filling materials, also known as bio-ceramics, still primarily consist of tricalcium silicate and dicalcium silicate.

Root canal treatment, also known as endodontic therapy, is a dental procedure aimed at treating pulp necrosis and root canal infections, with the main goals being bacterial removal, biofilm elimination, canal sealing to prevent infection, and promotion of healing of periapical tissues. Root canal treatment involves three stages: canal preparation, disinfection, and filling.

The performance and efficacy of root canal filling pastes are crucial in root canal treatment, playing a decisive role in reducing postoperative reactions and improving treatment success rates. Compared to zinc oxide-eugenol, calcium hydroxide, and resin-based sealers, bio-ceramic sealers demonstrate superior sealing properties and biocompatibility. Bio-ceramic sealers can release OH-ions continuously, exhibit a high pH value with strong antibacterial capabilities, possess good calcium ion release ability and root strengthening potential, are easy to handle by direct injection into the root canal, saving operation time and material costs. In recent years, with further research, it has been found that bio-ceramic sealers not only exhibit excellent sealing properties in root canal treatment but also demonstrate biological activities such as antibacterial, analgesic, and tissue repair promotion through interaction with periapical tissues.

Ideal requirements for clinical root canal filling materials are: 1) no leakage, providing a tight seal; 2) good bonding with root canal walls after curing; 3) radiopacity for visualization on X-rays; 4) fine particles with good flowability; 5) no shrinkage upon curing; 6) antibacterial properties; 7) appropriate curing time; 8) insolubility in tissue fluids; 9) good biocompatibility; 10) easy removal.

### SUMMARY

The object of the present disclosure is to provide the application of calcium aluminate in pre-mixed dental filling materials, pre-mixed dental filling materials, and their preparation methods. The present disclosure uses calcium aluminate as the main water-setting material, possessing characteristics of high temperature resistance, low microleakage, high adhesion, short water-setting time, and high strength after water-setting. Optionally, the pre-mixed dental filling materials of the present disclosure also include calcium oxide, calcium pyrophosphate, a radiopacifier, and polyethylene glycol of specific molecular weight.

A pre-mixed dental filling material includes calcium aluminate as a primary hydraulic setting material, where the filling material is non-aqueous.

Optionally, the pre-mixed dental filling material does not includes silicate materials.

Optionally, the calcium aluminate accounts for 15%-70% of the total mass of the pre-mixed dental filling material, such as 15%, 20%, 25%, 30%, 35%, 40%, 50%, 60%, 70%, etc.

Optionally, the filling material is applied in the fields of pulp capping, root canal treatment, and/or hard tissue repair.

Optionally, the filling material further includes calcium oxide, calcium pyrophosphate, a radiopacifier, and/or a water-miscible liquid carrier.

In the present disclosure, it has been found that the appropriate addition of calcium oxide in the filling material system provides the following benefits: 1) shortens the water-setting time; 2) causes slight expansion of the material volume after water-setting, preventing microleakage at the root apex, whereas a material without calcium oxide will shrink and crack after water-setting; 3) raises the pH value, acting as an antibacterial agent. Furthermore, it has been found that replacing calcium oxide with conventional material calcium hydroxide does not achieve the same benefits of shortening the water-setting time and causing slight expansion of the water-set material volume in the present disclosure.

Moreover, it has been found that using conventional phosphate materials such as tricalcium phosphate, hydroxyapatite, or dicalcium phosphate will not result in water-setting of the filling material according to ISO6876 method within 24 hours. Through material screening, it has been discovered that calcium pyrophosphate is suitable for the filling material system of the present disclosure without affecting the water-setting of the filling material.

Furthermore, it has been found that if tricalcium aluminate is used to replace calcium aluminate (Table 3, Formula 7), the filling material of the present disclosure does not water-set according to the ISO6876 method within 24 hours. Similarly, when a mixture of calcium aluminate and tricalcium aluminate is used as the main water-setting material (Table 3, Formula 8), the filling material of the present disclosure does not water-set according to the ISO6876 method within 2 hours. Therefore, in some embodiments, tricalcium aluminate is not used to replace or partially replace calcium aluminate.

In some embodiments, in terms of mass percentage, the filling material comprises the following components: 15%-70% calcium aluminate (e.g., 15%, 20%, 25%, 35%, 45%, 50%, 60%, 65%, 70%), 0.5%-10% calcium oxide (e.g., 0.5%, 1%, 2%, 5%, 10%), 5%-20% calcium pyrophosphate (e.g., 5%, 8%, 10%, 15%, 20%), 10%-30% radiopacifier (e.g., 10%, 15%, 20%, 25%, 28%, 30%), with the remainder being the liquid carrier.

Optionally, the radiopacifier is selected from one or more of zirconium hydroxide, bismuth hydroxide, zirconium oxide, bismuth oxide, with bismuth hydroxide being the preferred choice.

Optionally, the liquid carrier is selected from one or more of glycerol, propylene glycol, polyethylene glycol 200-600, with polyethylene glycol 400 being the preferred choice.

Furthermore, the present disclosure also includes a method for preparing pre-mixed dental filling materials, comprising the following steps:
A. Except for the liquid carrier component, place the other filling materials into anhydrous ethanol, thoroughly grind using a ball mill, and after evaporating the anhydrous ethanol, then set aside;
B. Place the mixture into the liquid carrier component, thoroughly grind in a ball mill to homogenize and disperse the components, and obtain the final product.

In conclusion, due to the adoption of the above technical solutions, the present disclosure has the following beneficial effects, which are not intended to limit the scope of the present application:
1. The pre-mixed dental filling material containing calcium aluminate has high flowability and can withstand high temperatures up to 200° C. without affecting the flowability of the formulation.
2. The pre-mixed dental filling material containing calcium aluminate can prevent leakage at the root apex, achieving a “tight seal” and avoiding issues with root apex leakage.
3. The pre-mixed dental filling material containing calcium aluminate exhibits excellent adhesive properties.
4. The water-setting time of the pre-mixed dental filling material containing calcium aluminate is approximately 2 hours.
5. The strength of the pre-mixed dental filling material containing calcium aluminate after water-setting, with a loading force of about 9.8N, is approximately 13-17HV, demonstrating excellent mechanical strength.

### BRIEF DESCRIPTION OF THE DRAWINGS

FIG. 1 shows the water-set SEM image of formulation 2 of the present disclosure.

FIG. 2 shows the elemental analysis spectrum of the water-set formulation 2 of the present disclosure.

FIG. 3 shows the water-set SEM image of a domestic commercial root canal filling paste.

FIG. 4 shows the water-set SEM image of the imported commercial root canal filling paste 1.

FIG. 5 shows the water-set SEM image of the imported commercial root canal filling paste 2.

FIG. 6 shows the microleakage performance of the formulations of the present disclosure compared with domestic and imported commercial root canal filling pastes.

### DETAILED DESCRIPTION

Below, combined with the drawings, a detailed description of the present disclosure is provided.

In order to clarify the objectives, technical solutions, and advantages of the present disclosure, the following detailed description is provided in combination with the drawings and embodiments. It should be understood that the specific embodiments described here are only for explaining the present disclosure and are not intended to limit the present disclosure.

### I. Water Setting and Strength Test

### 1. Test Materials

Formulations 1-7 pastes from Table 3, commercially available domestic root canal filling paste, commercially available imported root canal filling paste 1, and commercially available imported root canal filling paste 2.

### 2. Test Methods

S2.1. The water setting test method and criteria are based on ISO6876. After the materials are completely and thoroughly set, use SME to scan and photograph the surface of the set materials.
S2.2. Strength test method: Take the water-set materials, appropriately polish the surface, and apply a load of approximately 9.8N. After loading and unloading, obtain a diamond-shaped indentation and measure the distance between the two diagonal points of the indentation to automatically obtain the hardness value. Repeat the measurement five times for the same sample to obtain the average hardness value. For the material of formulation 6, apply a load of approximately 19.6N.

### S2.2.1. Test Instrument: Digital Vickers Hardness Tester (HVS-50 Type).

### 3. Test Results

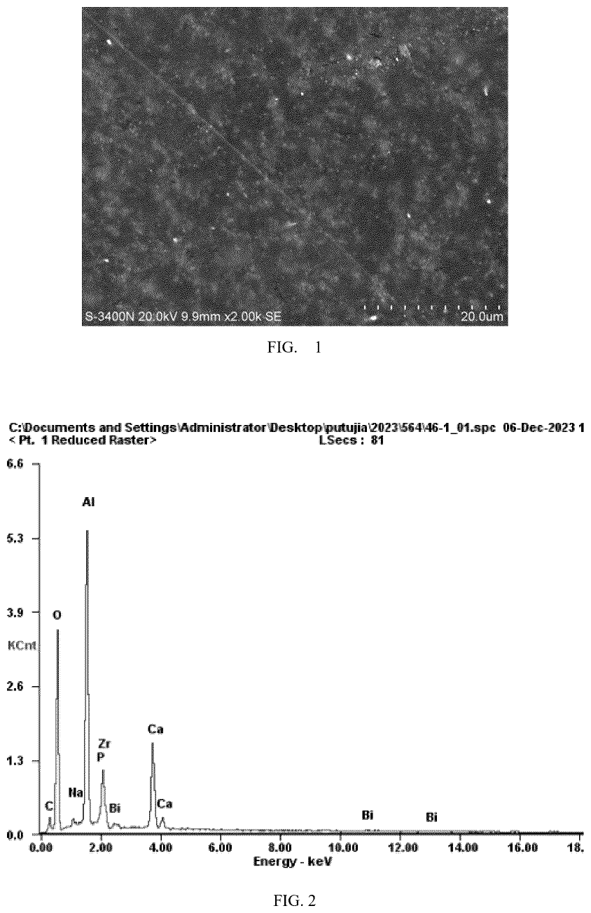

S3.1. Use SME to scan and photograph the surface of the water-set materials for formulation 2 paste, commercially available domestic root canal filling paste, commercially available imported root canal filling paste 1, and commercially available imported root canal filling paste 2 (see FIGS. 1, 3, 4, and 5).
S3.2. Use SME to conduct elemental analysis on the surface of the water-set material of formulation 2 paste (see FIG. 2).
S3.3. The time required for water setting and the hardness values after setting for each material are shown in Table 1.

TABLE 1
Water Setting Time and Hardness After Setting

| | Hardness | | | Hardness | |
|---|---|---|---|---|---|
| Product | Value | Time | Product | Value | Time |
| Formulation 1 | 12.25HV | 2 h | Formulation 2 | 16.15HV | 2 h |
| Formulation 3 | 16.53HV | 2 h | Formulation 4 | 16.92HV | 2 h |
| Formulation 5 | 17.87HV | 2 h | Formulation 6 | 24.78HV | 4 h |
| Formulation 7 | — | 24 h | Domestic | 9.12HV | 24 h |
| | | Un- | Paste | | |
| | | cured | | | |
| Imported | 16.23HV | 4 h | Imported | 15.51HV | 4 h |
| Paste 1 | | | Paste 2 | | |

Note:
“—” indicates that the hardness value was not measured due to the excessively slow water setting process of formulation 7.

### 4. Test Conclusions

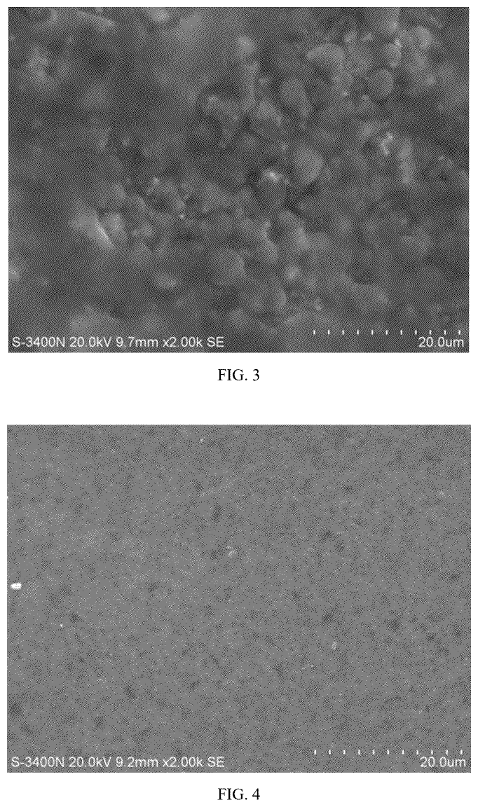

4.1. Using SME to scan and photograph the surface of the water-set materials (see FIGS. 1, 3, 4, 5), it can be concluded that the denser the surface, the more complete the hydration, and the smoother the surface. The hydration effect of formulation 2 paste (FIG. 1) is comparable to that of commercially available imported root canal filling paste 1 (FIG. 4), and superior to that of commercially available imported root canal filling paste 2 (FIG. 5) and commercially available domestic root canal filling paste (FIG. 3).
4.2. Based on the analysis of the water setting time and hardness values of each material in Table 1, the hardness of the water-set paste of this disclosure is comparable to that of the two commercially available imported root canal filling pastes and superior to that of the commercially available domestic root canal filling paste. The water setting time of the paste of this disclosure is the shortest.
4.3. According to Table 1, the water setting time and hardness after setting of formulations 1-7 pastes of this disclosure indicate that the appropriate addition of calcium oxide can shorten the water setting time of the paste of this disclosure. However, as the amount of calcium oxide increases, the strength of the water-set paste of this disclosure decreases.

### II. Microleakage Test

### 1. Test Materials

1.1 Paste formulations 2, 4, and 6 from Table 3, commercially available domestic root canal filling paste, commercially available imported root canal filling paste 1, and commercially available imported root canal filling paste 2.
1.2 Silver nitrate

### 2. Test Methods

2. Inject each group of pastes into butterfly needles, place the tip of the butterfly needle in a 5% silver nitrate solution, and soak for 7 days. After 7 days, observe the distance that the silver nitrate has leaked into the tip of the butterfly needle.

### 3. Test Results

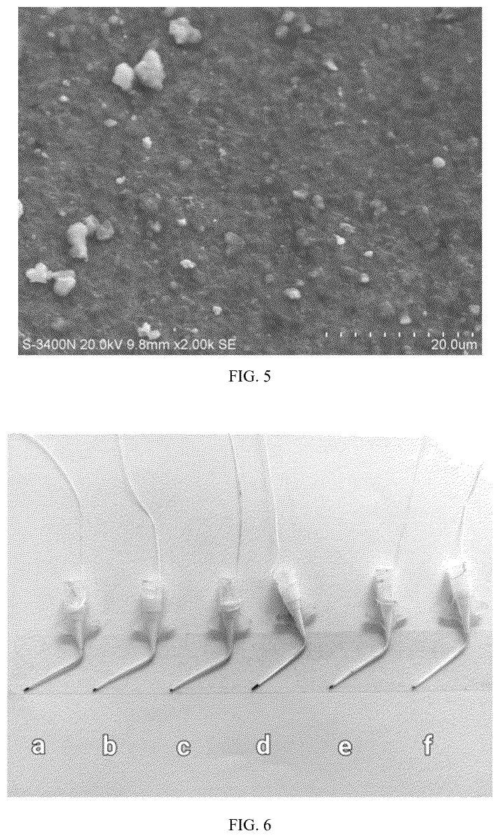

Paste formulation 2 (FIG. 6, c), paste formulation 4 (FIG. 6, b), paste formulation 6 (FIG. 6, a), commercially available domestic root canal filling paste (FIG. 6, d), commercially available imported root canal filling paste 1 (FIG. 6, f), and commercially available imported root canal filling paste 2 (FIG. 6, e).

### 4. Test Conclusion

As shown in FIG. 6, paste formulations 2 and 4 have leakage comparable to the two commercially available imported root canal filling pastes (less than 1 mm), and are superior to paste formulation 6 and the commercially available domestic root canal filling paste. Paste formulation 6 is slightly better than the commercially available domestic root canal filling paste in terms of leakage. From paste formulations 2 (FIG. 6, c), 4 (FIGS. 6, b), and 6 (FIG. 6, a), it can be seen that appropriately adding calcium oxide can reduce the apical microleakage of the paste formulations in this disclosure.

### III. Comparison of Flowability with Existing Products

### 1. Test Materials

Paste formulations 1, 2, 3, and 4 from Table 3, commercially available domestic root canal filling paste, commercially available imported root canal filling paste 1, and commercially available imported root canal filling paste 2.

### 2. Test Methods

According to ISO 6876 method.

### 3. Test Results

TABLE 2
Average Compression Diameter

| | Average Compression Diameter |
|---|---|
| | (measured 4 times in different |
| Product | directions and averaged) |
| Formulation 1 | 25 mm |
| Formulation 2 | 27 mm |
| Formulation 3 | 27 mm |
| Formulation 4 | 26 mm |
| Commercially available domestic | 12 mm |
| root canal filling paste | |
| Commercially available imported | 22 mm |
| root canal filling paste 1 | |
| Commercially available imported | 24 mm |
| root canal filling paste 2 | |

### 4. Test Conclusion

The flowability of the paste formulations in this disclosure is superior to that of existing commercially available products, exhibiting high flowability characteristics.

### IV. Specific Embodiments

### Example 1

The following raw materials are prepared according to the mass percentage: 15-70% calcium aluminate (CaA), 0.5-10% calcium oxide (CaO), 5-20% calcium pyrophosphate (CaPP), 10-30% radiation shielding agent, and the balance being polyethylene glycol 400 (PEG-400).

### Preparation Method:

1. Place the above raw materials into anhydrous ethanol, use a ball mill to grind them finely, and then evaporate the anhydrous ethanol for later use.
2. Put the finely ground components into PEG-400, grind them thoroughly in the ball mill to ensure the components are fully homogenized and dispersed to obtain the final product.

### Example 2

Table 3 lists the raw materials and their mass ratios for 10 formulations. For each formulation, take the raw materials according to their mass ratios and prepare a homogeneous paste using the preparation method described in Embodiment 1.

TABLE 3
Raw Materials and Their Mass Percentages (%)

| | | | | | | | PEG- |
|---|---|---|---|---|---|---|---|
| | CaA | Ca₃A | CaO | CaPP | ZrO | Bi(OH)₃ | 400 |
| Formulation 1 | 20 | — | 10 | 15 | 15 | 15 | 25 |
| Formulation 2 | 28 | — | 2 | 18 | 14 | 13 | 25 |
| Formulation 3 | 35 | — | 2 | 18 | 10 | 10 | 25 |
| Formulation 4 | 40 | — | 1 | 13 | 11 | 10 | 25 |
| Formulation 5 | 50 | — | 0.5 | 5 | 5 | 10 | 29.5 |
| Formulation 6 | 35 | — | — | 18 | 11 | 11 | 25 |
| Formulation 7 | — | 30 | 5 | 20 | 10 | 10 | 25 |
| Formulation 8 | 15 | 20 | 4 | 15 | 10 | 10 | 26 |
| Formulation 9 | 35 | — | 2 | 20 | 10 | 15 | 18 |
| Formulation 10 | 70 | — | 0.5 | 5.5 | — | 10 | 14 |

In Table 1, CaA: calcium aluminate; Ca₃A: tricalcium aluminate; CaO: calcium oxide; CaPP: calcium pyrophosphate; ZrO: zirconium oxide; Bi(OH)₃: bismuth hydroxide; PEG-400: polyethylene glycol 400.

PEG-400 in the formulations can be replaced with PEG-200 or PEG-600.

The above description is only a preferred embodiment of the present disclosure and is not intended to limit the disclosure. Any modifications, equivalent replacements, and improvements made within the spirit and principles of the present disclosure should be included within the scope of the present disclosure.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the patent protection of the present disclosure shall be defined by the appended claims.

What is claimed is:

1. A pre-mixed dental filling material, which is a non-aqueous material, the pre-mixed dental filling material comprising:
calcium aluminate as a primary hydraulic setting material.

2. The pre-mixed dental filling material according to claim 1, without silicate materials.

3. The pre-mixed dental filling material according to claim 1, wherein the calcium aluminate accounts for 15%-70% of the total mass of the pre-mixed dental filling material.

4. The pre-mixed dental filling material according to claim 1, wherein the filling material is used in the fields of pulp capping, root canal therapy, and/or hard tissue repair.

5. The pre-mixed dental filling material according to claim 1, further comprising calcium oxide, calcium pyrophosphate, a radiopacifier, and a water-miscible liquid carrier.

6. The pre-mixed dental filling material according to claim 5, wherein by mass percentage: the calcium aluminate accounts for 15%-70%, the calcium oxide accounts for 0.5%-10%, the calcium pyrophosphate accounts for 5%-20%, the radiopacifier accounts for 10%-30%, and the remainder is the water-miscible liquid carrier.

7. The pre-mixed dental filling material according to claim 5, wherein the radiopacifier is selected from zirconium hydroxide, bismuth hydroxide, zirconium oxide, or bismuth oxide, either alone or in combination.

8. The pre-mixed dental filling material according to claim 5, wherein the water-miscible liquid carrier is selected from glycerol, propylene glycol, or polyethylene glycol 200-600, either alone or in combination.

9. The pre-mixed dental filling material according to claim 1, further comprising calcium oxide.

10. The pre-mixed dental filling material according to claim 1, further comprising calcium pyrophosphate.

11. The pre-mixed dental filling material according to claim 1, further comprising radiopacifier.

12. The pre-mixed dental filling material according to claim 11, wherein the radiopacifier is selected from zirconium hydroxide, bismuth hydroxide, zirconium oxide, or bismuth oxide, either alone or in combination.

13. The pre-mixed dental filling material according to claim 1, further comprising a water-miscible liquid carrier.

14. The pre-mixed dental filling material according to claim 13, wherein the water-miscible liquid carrier is selected from glycerol, propylene glycol, or polyethylene glycol 200-600, either alone or in combination.

15. A method for preparing the pre-mixed dental filling material according to claim 1, the method comprising:
except for any liquid carrier components, placing other components of the filling material into anhydrous ethanol, using a ball mill to grind them finely, and evaporating the anhydrous ethanol to obtain a mixture for later use;
placing the mixture into a liquid carrier component, grinding it thoroughly in a ball mill to ensure that all components are homogeneously dispersed, and then obtain a final product.
